(19) United States
(12) Patent Application Publication
(10) Pub. No.: US 2022/0396841 A1
(43) Pub. Date: Dec. 15, 2022

(54) DETECTION AND ELIMINATION OF ABERRANT CELLS

(71) Applicant: Viome Life Sciences, Inc., Bellevue, WA (US)

(72) Inventor: Momchilo VUYISICH, Bothell, WA (US)

(21) Appl. No.: 17/779,439

(22) PCT Filed: Nov. 27, 2020
(86) PCT No.: PCT/US20/62495

§ 371 Date: May 24, 2022

Related U.S. Application Data
(60) Provisional application No. 62/942,728, filed on Dec. 2, 2019.

(51) Int. Cl. C12Q 1/6886

(52) U.S. Cl. CPC C12Q 1/6886; C12Q 2600/158; C12Q 2600/156; C12Q 2600/106; C12Q 1/6869

(57) ABSTRACT

Provided herein are methods for detecting cells in a subject that express aberrant proteins. Methods are also provided for eliminating such cells expressing aberrant proteins.

### REFERENCE TO RELATED APPLICATIONS

This application claims the benefit of the priority date of U.S. provisional application 62/942,728, filed Dec. 2, 2019, the contents of which are incorporated herein in their entirely.

### STATEMENT AS TO FEDERALLY SPONSORED RESEARCH

None.

### THE NAMES OF THE PARTIES TO A JOINT RESEARCH AGREEMENT

None.

### SEQUENCE LISTING

None.

### BACKGROUND

Aberrant cells, including pathological cells and senescent cells, can express aberrant proteins which contribute to aberrant functioning of the cells. As aberrant cells increase in abundance in a tissue, the tissue can show signs of unhealthy functioning, and can lead to aging and death.

Aberrant forms of proteins are known to occur and can include forms acquired by somatic mutation, such as splice variants or acquired sequence variations.

### SUMMARY

In one aspect provided herein is a method comprising: a) providing a tissue sample comprising aberrant cells from a subject; and b) detecting, in the sample, the expression of at least one aberrant protein. In one embodiment the aberrant protein is an aberrant structural protein. In another embodiment the aberrant protein is selected from: a splice variant, a genetic variant, a glycosylation variant and a lipidation variant. In another embodiment the aberrant protein is encoded by a genetic variant selected from a single nucleotide polymorphism (SNP), an insertion, a deletion, a gene fusion, a transversion, and a gene truncation. In another embodiment the aberrant protein is a housekeeping protein. In another embodiment the aberrant protein is not encoded by an oncogene or a tumor suppressor gene. In another embodiment the aberrant protein is not a transmembrane protein. In another embodiment the subject is a human or a non-human animal. In another embodiment the tissue sample comprises a preponderance of senescent cells. In another embodiment the tissue is derived from an organ system selected from muscular system, digestive system, respiratory system, urinary system, reproductive system, endocrine system, circulatory system, nervous system, and integumentary system. In another embodiment the sample is a solid tissue biopsy sample (e.g., a needle aspirate), a bone marrow biopsy sample or a liquid biopsy sample. In another embodiment detecting comprises detecting an aberrant polypeptide. In another embodiment the aberrant polypeptide is detected by mass spectrometry. In another embodiment mass spectrometry comprises multiple reaction monitoring (MRM) mass spectrometry. In another embodiment detecting comprises detecting mRNA encoding the aberrant protein. In another embodiment detecting mRNA comprises sequencing cDNA prepared from the mRNA. In another embodiment comprises sequencing genomic DNA encoding the protein. In another embodiment detecting comprises detecting a plurality of aberrant forms of a protein. In another embodiment detecting comprises detecting an aberrant form or forms of each of a plurality of proteins. In another embodiment detecting comprises determining a measure of the aberrant protein in the sample. In another embodiment the measure is selected from presence or absence, an absolute amount, and a relative amount (e.g., an amount of the aberrant protein compared to an amount of the normal protein). In another embodiment the method further comprises determining whether the measure is above a threshold measure. In another embodiment the threshold is a function, e.g., a positive function, of the subject's chronological age. In another embodiment detecting comprises determining a fraction of cells in the tissue sample expressing the aberrant protein. In another embodiment no more than 1% of cells in the tissue are malignant. In another embodiment the method further comprises: c) inferring, from the quantitative measure, a biological age of the tissue. In another embodiment the method further comprises: c) outputting the quantitative measure to an electronic device accessible by the subject.

In another aspect provided herein is a method of eliminating, in a subject, aberrant cells expressing at least one aberrant protein comprising: a) selecting a subject having aberrant cells that express the aberrant protein; and b) administering to the subject an immunotherapy to eliminate the aberrant cells. In one embodiment the subject has aberrant cells in an organ system selected from muscular system, digestive system, respiratory system, urinary system, reproductive system, endocrine system, circulatory system, nervous system, and integumentary system. In another embodiment the subject is identified by a method as disclosed herein. In another embodiment a biopsy sample from the subject detects the presence of aberrant cells expressing the aberrant protein. In another embodiment the immunotherapy comprises administering to the subject an antibody that binds to a fragment of a protein, wherein the fragment comprises an aberrant amino acid sequence presented by an MHC molecule on a surface of the cell. In another embodiment the antibody is an antibody-drug conjugate. In another embodiment the immunotherapy comprises training of APC cells with the aberrant proteins. In another embodiment the immunotherapy comprises eliciting an immune response by administering the aberrant protein form to the subject. In another embodiment the immunotherapy comprises administering a DNA vaccine comprising DNA encoding the aberrant protein or an aberrant fragment thereof. In another embodiment the immunotherapy comprises administering to the subject a CAR-T cell that recognizes a cell expressing the aberrant form of the protein. In another embodiment the aberrant cell is a senescent cell or a pathological cell. In another embodiment the aberrant cell is not a malignant cell. In another embodiment the aberrant protein is not a transmembrane protein, membrane-anchored protein or a protein encoded by an oncogene or a tumor suppressor gene.

In another aspect provided herein is an article comprising: a) one or a plurality of solid supports, each solid support comprising a nucleic acid probe that binds to a nucleic acid having a nucleotide sequence of a senescence-associated polypeptide.

In another aspect provided herein is a composition comprising: a) one or a plurality of pairs of oligonucleotide primers, each pair of primers adapted for amplifying a nucleic acid having a nucleotide sequence of a senescence-associated polypeptide.

In another aspect provided herein is a composition comprising: a) for each of one or a plurality of senescence-associated polypeptides, a stable isotope standard polypeptide.

In another aspect provided herein is a method comprising: a) providing a set of biological samples from each of plurality of subjects wherein the subjects comprise a plurality of subjects from each of a plurality of different functional states; b) performing -omic analysis on each of the biological sample to produce an -omic data training dataset; and c) training a machine learning algorithm on the -omic data training data set to generate a mapping function that infers an aberrant functional state from the -omic data. In one embodiment the functional state is a stage of senescence. In another embodiment the stages of senescence span a time period of at least one year, at least 3 years, at least 5 years, at least 10 years, at least 20 years, at least 30 years, at least 40 years, at least 50 years, at least 60 years, at least 70 years, at least 80 years, at least 90 years, or at least 100 years. In another embodiment, within the span of time, the plurality of ages are separated by no more than 6 months, one year, five years, ten years or 20 years. In another embodiment, at least 3 ages are separated by no more than 3 years, 6 years, 9 years or 15 years. In another embodiment each of the ages comprise at least any of 10, 25, 50, 100 or 200 subjects. In another embodiment the different functional states comprise different health states. In another embodiment the health states comprise healthy and unhealthy functioning of an organ or an organ system. In another embodiment the biological samples comprise samples from a tissue selected from epithelial, connective, nervous and muscle tissue. In another embodiment the -omic data is selected from genomic, epigenomic, transcriptomic, proteomic, metabolomic, lipidomic, glycomic, immunomic, phenomic and exposomic. In another embodiment the -omic data is blood transcriptomic data. In another embodiment performing -omic analysis comprises sequencing nucleic acids in the sample. In another embodiment the nucleic acids comprise genomic DNA and/or mitochondrial DNA. In another embodiment the nucleic acids comprise RNA. In another embodiment the RNA comprises mRNA. In another embodiment performing -omic analysis comprises bifulfite treatment of nucleic acids and sequencing of the bisulfite-treated nucleic acids. In another embodiment performing -omic analysis comprises separating and identifying proteins in the sample. In another embodiment separating and identifying is performed by mass spectrometry. In another embodiment performing -omic analysis comprises sequencing polysaccharides in the sample. In another embodiment performing -omic analysis comprises measuring metabolites in the sample. In another embodiment the mapping function is a classifier. In another embodiment the mapping function is a regressor. In another embodiment the mapping function uses measures of aberrant nucleic acid sequences. In another embodiment the mapping function uses measures of aberrant proteins. In another embodiment the mapping function uses measures of aberrant complex carbohydrates. In another embodiment the classifier uses gene expression information to infer functional state.

In another aspect provided herein is a method comprising: a) providing a biological sample from a subject; b) performing -omic analysis on the biological sample to produce an -omic data test dataset; c) executing a mapping function on the -omic data to infer a chronological age based on the -omic data; d) outputting the inferred age to an electronic device accessible by the subject.

In another aspect provided herein is a method comprising: a) providing a biological sample from a subject; b) performing -omic analysis on the biological sample to produce an -omic data test dataset; c) executing a mapping function on the -omic data to infer function or dysfunction of one or more organs in the subject based on the -omic data; and d) outputting the inferred age to an electronic device accessible by the subject.

In another aspect provided herein is a method comprising: a) providing a chronological age and biological sample from a subject; b) performing -omic analysis on the biological sample to produce an -omic data test dataset; c) executing a mapping function on the -omic data to infer an age based on the -omic data; d) determining that a difference between the chronological age and the inferred age is above a predetermined threshold; and e) administering to the subject an (organ-specific) immunotherapy to destroy cells expressing the aberrant proteins.

### DETAILED DESCRIPTION

### I. Introduction

Disclosed herein are, among other things, machines, articles, and methods for detecting aberrant cells and eliminating (e.g., destroying, killing or removing) those cells from an animal subject.

Detection and elimination of aberrant cells would improve function of tissues and organs in otherwise healthy or in senescent individuals Elimination of such cells, and possible replacement by healthy (functional) cells, is expected to result in more healthy functioning of organs and slower aging process. The cells that are removed are expected to be replaced by functional cells derived from stem cells (either naturally occurring or introduced by an intervention).

### II. Aberrant Cells

Aberrant cells are cells that express an unhealthy phenotype. An unhealthy phenotype may be seen as diminished or pathological (diminished function) or as senescence (sometimes referred to as a diminished phenotype, a pathological phenotype or a senescent phenotype). As a reference, an unhealthy phenotype of a cell can be determined as compared with the phenotype of the cell type in a healthy individual organism at physical and sexual maturity, and before onset of senescence. In humans, this includes adults between about 18 and 28 years of age. In animals, comparable metrics can be used. For example, many dog breeds reach maturity at about one year of age and begin showing signs of senescence at about age 5 or 6. Horses may reach maturity at around age 3 to around age 4, and show signs of aging around age 10 or 12. This approach can be applied to any living organism. Cells of pre-adult individuals, who may be growing, are generally not considered unhealthy as used herein.

The presence of unhealthy cells can manifest as pathological or diminished functioning of tissues or organs of which the cells are a part. For example, diminished functioning of the heart can be measured as decrease maximum heart rate. Diminished muscle functioning can be measured as decreased strength. Diminished functioning of liver can be measured as reduced ability to process toxins. Diminished lung function can be measured as diminished lung capacity. Diminished kidney function can be measured as reduced ability to produce urine and remove waste from the blood. Diminished intestinal function can be measured as reduced ability to absorb nutrients and increased occurrence of pathologies, such as leaky gut or diverticulitis. Diminished brain function can be measured by reduced short term memory or processing speed.

At the cellular level, any measure of pathology used by pathologists can be used to detect unhealthy cells. This includes histologic methods, such as microscopic examination of stained tissue of fixed cells, flow cytometry, enzyme histochemistry, immunochemistry, and molecular biological methods such as microarray, sequencing, etc. Signs of pathology include, for example, hyperplasia, hypertrophy, atrophy, and metaplasia (e.g., malignancy or cancer).

A senescent cell can be a cell with diminished functioning that does not otherwise show signs of pathology. This diminished functionality contributes the phenomenon we refer to as “aging”. Such cells may not be subject to apoptosis and may not be replaced by stem cells in an organ. For purposes of this disclosure, a senescent cell is considered to be an aberrant cell. During their life, senescent cells may begin to express aberrant proteins that diminish healthy functioning of the cell.

### III. Aberrant Proteins

Aberrant cell functioning can result from the expression of proteins with aberrant structures (“aberrant structural proteins”) and/or aberrant expression (i.e., over-expression or under expression) (“aberrantly expressed proteins”), together referred to as “aberrant proteins”. The presence of aberrant proteins can confer an unhealthy phenotype on a cell. An aberrant structural protein can result from a particular primary, secondary, tertiary or quaternary structure, or post-translational modification of the protein, that negatively alters the functioning of the protein. An aberrantly expressed protein results from aberrant expression of the protein, e.g., over-expression or under-expression. Accordingly, an unhealthy phenotype can be inferred by the presence of a protein with an aberrant structure or the presence of an aberrant abundance of an aberrant protein or an otherwise normal protein.

Aberrant proteins can be encoded in the germline. Alternatively, they can result from somatic mutation and generally increase as organisms age.

Aberrant proteins can occur from a number of different mechanisms. They may result from errors at the genetic level in the gene encoding the protein, for example, from a single nucleotide polymorphism (SNP), an insertion, a deletion, a gene fusion, a transversion, or a gene truncation. Such errors contribute to changes in the primary structure (amino acid sequence) of the resulting polypeptide. Aberrations may manifest as splice variants, in which an RNA transcript is differently spliced in the production of mRNA. Such aberrations can result from changes at the DNA sequence level, or as a result of changes in function of RNA splicing enzymes. Aberrations can occur as a result of RNA editing that accumulates over time, whether it is pre-programmed or not.

Aberrations can occur at the higher structural level as a result of mis-folding, which interrupts secondary structure, or impaired to form tertiary structures such as alpha helix or beta sheet, or impaired ability to interact with other proteins in quaternary structure. For example, a change in the DNA or RNA sequence, or post-translational modification can affect protein stability, protein-ligand interactions, and/or catalytic abilities. In some cases, the function of the protein can be enhanced and in others, impaired. Each phenomenon can lead to the overall impaired function of the cell.

Aberrations also can be seen at the post-translational modification level, for example, improper glycosylation, lipidation or phosphorylation.

Aberrant proteins can be aberrant forms of housekeeping proteins, that is, proteins encoded by genes that are constitutively expressed under normal conditions and are required for the maintenance of basic cellular function. Housekeeping genes include, for example:

(1) genes involved in gene expression (e.g., transcription factors, repressors, RNA splicing, translation factors, tRNA synthesis, RNA binding proteins, ribosomal proteins, mitochondrial ribosomal proteins, RNA polymerases, RNA splicing proteins, protein processing enzymes, heat shock proteins, histones, cell cycle regulating proteins, genes involved in apoptosis, oncogenes, and DNA repair/replication enzymes);
(2) genes involved in metabolism (e.g., carbohydrate metabolism enzymes, citric acid cycle enzymes, lipid metabolism enzymes, amino acid metabolism enzymes, NADH dehydrogenase, cytochrome C oxidase, ATPase, lysosome, proteasome 2.0 ribonuclease, and thioreductase);
(3) genes encoding structural proteins (e.g., cytoskeletal, organelle synthesis, and mitochondrion);
(4) genes encoding surface proteins (e.g., cell adhesion proteins, channel and transporter proteins, receptors, and HLA/immunoglobulin/cell recognition proteins); and
(5) genes encoding kinases and signaling (e.g., growth factors, tissue necrosis factor, and casein kinase).

Aberrant proteins for analysis can also include, for example, tissue specific proteins, such as those expressed in brain, liver, kidneys, etc.

Aberrant proteins can be detected and analyzed at a plurality of different “-omic” levels, including genomic, transcriptomic, proteomic, glycomic, lipidomic and phosphoryomic levels.

A. Protein Targets

In certain embodiments, certain proteins can be targeted for detection of aberrant forms. The proteins may be those known or identified to contribute to cell dysfunction in certain aberrant forms. They also can be tissue-specific, that is, proteins known to be commonly aberrant (and contributing to cell disfunction) in a particular cell type.

Such aberrant proteins can be identified as follows. In a particular tissue type in population of individuals of different ages, one would determine the identity and quantity of aberrant proteins. This would indicate proteins that tend to become aberrant over time, as well as the relative number of accumulated aberrations over time. Such proteins can be the target of detection. Furthermore, one could engineer healthy cells to incorporate these aberrant forms, either one-by-one or in combination. Effects on cell function can be examined to determine the effect on cell function of each aberrant or combination of forms.

### IV. Methods of Detecting the Presence of Aberrant Proteins

A. Subjects

Aberrant proteins can be detected in biological samples from a subject. As used herein, the term “subject” refers to an individual organism, e.g., an animal or a plant. Animal subjects include, without limitation, human and nonhuman multicellular animals. Non-human animals may be non-human mammals, birds, fish, reptiles and invertebrates. Non-human animals include, for example, bovines, swine, horses, sheep, goats, chickens, turkeys, dogs, cats and birds.

B. Biological Samples

Biological samples used in the detection of aberrant proteins include samples sourced from one or more organs or one or more tissues. A sample can comprise a plurality of tissues from an organ, or a single tissue type. The starting material can be the tissue, itself, cells isolated from a tissue or a homogenate of a tissue.

1. Organs and Tissues

Organs and tissues which are the source of biological samples used in the methods disclosed herein can be from any organ system of a subject.

Tissues can be sourced from an organ system selected from muscular system, digestive system, respiratory system, urinary system, reproductive system, endocrine system, circulatory system, nervous system, and integumentary system.

Tissues from the muscular system can be selected from skeleton, cartilage, ligaments, tendons and muscles.

Tissues from the digestive system can be selected from mouth, teeth, tongue, salivary glands, parotid glands, submandibular glands, sublingual glands, pharynx, esophagus, stomach, small intestine, duodenum, jejunum, ileum, large intestine, liver, gallbladder, mesentery, pancreas, anal canal and anus.

Tissues from the respiratory system can be selected from nasal cavity, pharynx, larynx, trachea, bronchi, lungs, and diaphragm.

Tissues from the urinary system can be selected from kidneys, ureter, bladder, and urethra.

Tissues from the reproductive system can be selected from, the female reproductive system, ovaries, fallopian tubes, uterus, vagina, vulva, clitoris, placenta, male reproductive system, testes, epididymis, vas deferens, seminal vesicles, prostate, bulbourethral glands, penis, and scrotum.

Tissues from the endocrine system can be selected from pituitary gland, pineal gland, thyroid gland, parathyroid glands, adrenal glands, and pancreas.

Tissues from the circulatory system can be selected from arteries and veins, heart, patent foramen ovale, arteries, veins, capillaries, and lymphatic system. It also includes blood, which is a connective tissue.

Tissues from the lymphatic system can be selected from lymphatic vessel, lymph node, bone marrow, thymus, spleen, gut-associated lymphoid tissue, and tonsils.

Tissues from the nervous system can be selected from, brain, cerebrum, cerebral hemispheres, diencephalon, the brainstem, midbrain, pons, medulla oblongata, cerebellum, the spinal cord, ventricular system, choroid plexus, peripheral nervous system, nerves, cranial nerves, spinal nerves, ganglia, and enteric nervous system.

Tissues from the sensory system can be selected from eye, cornea, iris, ciliary body, lens, retina, ear, outer ear, earlobe, eardrum, middle ear, ossicles, inner ear, cochlea, vestibule of the ear, semicircular canals, olfactory epithelium, tongue, and taste buds.

Tissues from the integumentary system can be selected from mammary glands, skin, and subcutaneous tissue.

2. Mature and/or Nonmalignant Tissue

A tissue sample can be a mature tissue. As used herein, a mature tissue is a tissue from an adult that has reached physical and sexual maturity. Such tissues typically are not undergoing growth, for example, growth associated with organogenesis. For example, a mature tissue is a tissue in a person who is past puberty. A tissue also can be a senescent tissue. As used herein, a senescent tissue is a tissue from an adult subject past his or her physical peak, e.g., for a human, someone who is over the age of 30, 40, 50, 60, 70, 80 or 90. In such subjects, a majority of the cells in the sample have reached their Hayflick limit, e.g., are unable to sustain further cell division due to, for example, to shortening of telomeres. Although, in certain conditions, such as progeria, humans can exhibit senescence at an early age, i.e., during youth and before maturity.

A tissue sample can be from a person more than any of 20, 30, 40, 50, 60, 70, 80, 90 or 100 years of age. A tissue sample can be from a person within one or two decades of 20, 25, 30, 35, 40, 45, 50, 55, 60, 65, 70, 75, 80, 85, 90, 95, 100 or 105 years of age (by way of example, between 30 and 40, or 35 and 45 (one decade) or between 30 and 50 or 35 and 55 (two decades)).

A tissue sample can be a non-malignant tissue sample. As used herein, a non-malignant tissue sample is a sample that a pathologist of ordinary skill in the art would not classify as malignant or make a diagnosis of cancer. In genetic testing of the tissue, any genetic alterations associated with cancer may be present below detectable levels. The term “malignant cell” refers to a cancer cell exhibiting uncontrolled cell division.

3. Provision

Samples derived from tissues can be provided by removing a sample of tissue from a subject. Such methods are well known in the art and include, for example, biopsy methods. Biopsies can be solid tissue biopsies or liquid biopsies. Solid tissue biopsies include, for example, needle biopsies, such as fine needle aspirates. A needle is inserted into a target organ or tissue and tissue is removed through the needle lumen. Liquid biopsy generally refers to the collection of a liquid sample from a subject which contains cells to be the subject of analysis. For example, a blood draw will contain cells present in the blood, in particular, cells of the hematopoietic system such as those of myeloid or lymphoid origin.

Liquid biopsy material also can contain cell free DNA.

Typically, analysis comprises homogenization of a tissue sample and isolation of molecules of interest from the homogenate. However, analysis also can involve isolation of one or a plurality of cells from the tissue for analysis.

C. Methods of Detection

Aberrant proteins can be detected or measured at any “omic” level, e.g., genomic, transcriptomic, proteomic, glycomic or phosphoromic levels. That is to say, one can detect genes encoding aberrant proteins, mRNA encoding aberrant proteins, aberrant proteins, or post-translational modifications of proteins.

1. Genomic Detection

Genes encoding aberrant proteins can be detected by sequencing genomic DNA. Genomic sequencing can involve whole genome sequencing. Alternatively, sequencing can involve isolating nucleic acids that encode target proteins suspected to harbor aberrations.

Nucleic acids can be isolated from the sample by any means known in the art. Polynucleotides can be isolated from a sample by contacting the sample with a solid support comprising moieties that bind nucleic acids, e.g., a silica surface. For example, the solid support can be a column comprising silica or can comprise paramagnetic silica beads. After capturing nucleic acids in a sample, the beads can be immobilized with a magnet and impurities removed. In another method, nucleic acids can be isolated using cellulose or polyethylene glycol.

DNA can be isolated with silica, cellulose, or other types of surfaces, e.g., Ampure SPRI beads. Kits for such procedures are commercially available from, e.g., Promega (Madison, Wis.) or Qiagen (Venlo, Netherlands).

a) Enriching for Target Molecules

DNA isolated from a cell can be enriched for polynucleotides encoding for target proteins such as those suspected of harboring aberrations. Nucleic acids can be enriched using probes having nucleotide sequences that can hybridize to target sequences. Such probes are typically coupled to a solid support. So, for example, the capture agent can comprise a polynucleotide probe attached to biotin. The probes can be, for example, at least 100 nucleotides long. Isolated DNA can be denatured and single strands having appropriate sequences can be hybridized to the probes. If the biotin probes are immobilized, non-hybridized nucleic acids can be removed and the target nucleic acids, captured by the probes, can be isolated. The isolated, enriched nucleic acids can be subject to amplification, for example, by PCR.

The amplified nucleic acids are generally sequenced for subsequent analysis. The methods described herein generally employ high throughput sequencing methods. As used herein, the term “high throughput sequencing” refers to the simultaneous or near simultaneous sequencing of thousands of nucleic acid molecules. High throughput sequencing is sometimes referred to as “next generation sequencing” or “massively parallel sequencing.” Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing (Complete Genomics), Heliscope single molecule sequencing, single molecule real time (SMRT) sequencing (PacBio), and nanopore DNA sequencing (e.g., Oxford Nanopore).

2. Transcriptomic Detection

Aberrant nucleic acid transcripts can be detected by nucleic acid sequencing. Such methods typically involve isolating messenger RNAs from cells in the sample, converting them, e.g., by reverse transcription, into cDNA and sequencing the cDNA.

In the case of RNA, the sample can be exposed to an agent that degrades DNA, for example, a DNase. Commercially available DNase preparations include, for example, DNase I (Sigma-Aldrich), Turbo DNA-free (ThermoFisher) or RNase-Free DNase (Qiagen). Also, a Qiagen RNeasy kit can be used to purify RNA. In another embodiment, a sample comprising DNA and RNA can be exposed to a low pH, for example, pH below pH 5, below pH 4 or below pH 3. At such pH, DNA is more subject to degradation than RNA.

Messenger RNA can be enriched from a sample using polynucleotide probes, as described above. In this case, the probes can comprise a poly-T sequence that hybridizes to the poly-A tail of RNA.

RNA can be converted into cDNA by reverse transcription, and cDNA can be sequenced by the methods as described above.

3. Target Species

Determination of target species depends on the particular application. In the case of transcriptome analysis, target species can include microbial and/or host mRNA. In the case of microbiome analysis, target species may include bacterial rRNA used to identify microorganisms in the sample. In the case of genomic analysis, target species may include a selected set of genes of interest, e.g., genes associated with genetic diseases of predisposition to them, oncogenes, ancestry informative markers or short tandem repeat loci.

4. Proteomic Detection

Aberrant proteins can be detected directly by any means known in the art.

Proteins in a sample can be detected by mass spectrometry. Mass spectrometers typically include an ion source to ionize analytes, and one or more mass analyzers to determine mass. Mass analyzers can be used together in tandem mass spectrometers. Ionization methods include, among others, electrospray or laser desorption ionization. Mass analyzers include quadrupoles, ion traps, time-of-flight instruments and magnetic or electric sector instruments. In certain embodiments, the mass spectrometer is a tandem mass spectrometer (e.g., “MS-MS”) that uses a first mass analyzer to select ions of a certain mass and a second mass analyzer to analyze the selected ions. One example of a tandem mass spectrometer is a triple quadrupole instrument, the first and third quadrupoles act as mass filters, and an intermediate quadrupole functions as a collision cell. Mass spectrometry also can be coupled with up-stream separation techniques, such as liquid chromatography or gas chromatography. So, for example, liquid chromatography coupled with tandem mass spectrometry can be referred to as “LC-MS-MS”.

Mass spectrometers useful for the analyses described herein include, without limitation, Altis™ quadrupole, Quantis™ quadrupole, Quantiva™ or Fortis™ triple quadrupole from ThermoFisher Scientific, and the QSight™ Triple Quad LC/MS/MS from Perkin Elmer.

Selected reaction monitoring is a mass spectrometry method in which a first mass analyzer selects a polypeptide of interest (precursor), a collision cell fragments the polypeptide into product fragments and one or more of the fragments is detected in a second mass analyzer. The precursor and product ion pair is called an SRM “transition”. The method is typically performed in a triple quadrupole instrument. When multiple fragments of a polypeptide are analyzed, the method is referred to as Multiple Reaction Monitoring Mass Spectrometry (“MRM-MS”). Typically, protein samples are digested with a proteolytic enzyme, such as trypsin, to produce peptide fragments. Heavy isotope labeled analogues of certain of these peptides are synthesized as standards. These standards are referred to as Stable Isotopic Standards or “SIS”. SIS peptides are mixed with a protease-treated sample. The mixture is subjected to triple quadrupole mass spectrometry. Peptides corresponding to the SIS standards are detected with high accuracy. Peptides can be synthesized to order, or can be available as commercial kits from vendors such as, for example, e.g., ThermoFisher (Waltham, Mass.) or Biognosys (Zurich, Switzerland).

Accordingly, detection of a protein target by MRM-MS involves detection of one or more peptide fragments of the protein, typically through detection of a stable isotope standard peptide against which the peptide fragment is compared.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods and compositions disclosed herein. Suitable peptide MS and MS/MS techniques and systems are known in the art (see, e.g., Methods in Molecular Biology, vol. 146: “Mass Spectrometry of Proteins and Peptides”, by Chapman, ed., Humana Press 2000; Kassel & Biemann (1990) Anal. Chem. 62:1691-1695; Methods Enzymol 193: 455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more peptides. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS).

As used herein, the terms “multiple reaction monitoring (MRM)” or “selected reaction monitoring (SRM)” refer to a MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, the assay can include standards that correspond to the analytes of interest (e.g., peptides having the same amino acid sequence as that of analyte peptides), but differ by the inclusion of stable isotopes. Stable isotopic standards (SIS) can be incorporated into the assay at precise levels and used to quantify the corresponding unknown analyte. Additional levels of specificity are contributed by the co-elution of the unknown analyte and its corresponding SIS, and by the properties of their transitions (e.g., the similarity in the ratio of the level of two transitions of the analyte and the ratio of the two transitions of its corresponding SIS).

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS); APPI-MS/MS; and APPI-(MS)n. Peptide ion fragmentation in tandem MS (MS/MS) arrangements can be achieved using techniques known in the art, such as, e.g., collision induced dissociation (CID). As described herein, detection and quantification of biomarkers by mass spectrometry can involve multiple reaction monitoring (MRM), such as described, inter alia, by Kuhn et al. (2004) Proteomics 4:1175-1186. Scheduled multiple-reaction-monitoring (Scheduled MRM) mode acquisition during LC-MS/MS analysis enhances the sensitivity and accuracy of peptide quantitation. Anderson and Hunter (2006) Mol. Cell. Proteomics 5(4):573-588. Mass spectrometry-based assays can be advantageously combined with upstream peptide or protein separation or fractionation methods, such as, for example, with the tandem column system described herein.

### V. Building and Using Models to Identify Aberrant Proteins and to Classify Samples or Individuals as Having Aberrant Proteins.

Models can be created by statistical methods, including, for example, methods performed by machine learning. Machine learning involves training machine learning algorithms on training data sets comprising data from a plurality of test subjects.

Machine learning can be used to develop classifiers for classifying a sample from a subject as possessing aberrant proteins or as exhibiting signs of aberrant function, such as pathology or senescence. Machine learning methods involve building a training dataset including data to be used in the classifier, such as protein sequences of proteins in a sample, using the training dataset to develop a classifier to classify or infer a state for a categorical variable, such as pathology or senescence, and then using the classifier on a test dataset from a sample from a subject to classify the sample and/or make inferences. Because sequences of aberrant proteins will be variables used by any such classifier to distinguish healthy from unhealthy states, these aberrant proteins can, themselves, be targets for elimination from a subject by the methods described herein. Accordingly, provided herein are methods of inferring a health state or functional state of a subject or an organ or tissue of a subject. The methods can involve using a machine learning algorithm to develop a classifier based on -omic data, in particular, transcriptomic data, from samples from subjects belonging to both healthy and unhealthy states, and applying the classifier to data from a subject to be tested.

In some embodiments, the functional state of the subject is biological age. Biological age can be set in terms of years (e.g., within a range of 1, 2, 3, 4 of 5 years) or in terms of decades (e.g., spanning a ten year period, such as 30-40, or 55-65).

Also provided herein are methods of inferring biological age, e.g., senescence, in a subject. Methods involve using classifiers or regression to assign a biological (or functional) age for any tissue, organ, or whole organism in a subject based on amounts of one or a variety of aberrant proteins in the tissue, organ, or entire organism.

A. Training Dataset

Methods of generating models to infer healthy or unhealthy states, or to predict biological age or senescence, can involve providing a training dataset on which a machine learning algorithm can be trained to develop one or more models or classifiers to make an inference or to classify the state. The training dataset will include a plurality of training examples, typically for each of a plurality of subjects and typically in the form of a vector. Each training example will include a plurality of features and, for each feature, data, e.g., in the form of numbers or descriptors. Where learning is to be supervised, the data will include a classification of the subject into a category of a categorical variable to be inferred. For example, the categorical variable may be “disease state” and the categories or classifications of this variable can be “present” and “absent”. Typically, for machine learning, the training examples will have at least 10, at least 100, at least 500 or at least 1000 different features. The features selected are those on which prediction will be based. Typically, also, the training dataset will comprise at least 50, at least 100, at least 500 or at least 1000 subjects or training examples, and will include a plurality of examples from each possible state of the categorical variable.

A measurement of a variable can be any combination of numbers and words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category, such a “healthy” or “unhealthy”, “old” or “young”, “happy or “unhappy”, “subject 1 . . . subject n.” Measurements of a variable on an ordinal scale produce a ranking, such as “first”, “second”, “third”; or “youngest” to “oldest”, or order from most to least. Measurements on a ratio scale include, for example, any measure on a pre-defined scale, such as mass, signal strength, concentration, age etc.; as well as statistical measurements such as frequency, mean, median, standard deviation, or quantile. Measurements on a ratio scale can be normalized measures.

Values for features in the dataset can be quantitative measures of the feature or descriptive terms. Quantitative measures can be given as a discrete or continuous range. Examples of quantitative measures include a number, a degree, a level, a range or bucket. A number can be a number on a scale, for example 1-10. Alternatively, the score can embrace a range. For example, ranges can be high, medium and low; severe, moderate and mild; or actionable and non-actionable. Buckets can comprise discrete numerals, such as 1-3, 4-6 and 7-10.

1. Subject Data

Each subject used as a training example will typically include the following types of information. In certain embodiments, a training set includes, for each individual, transcriptomic data, such as blood transcriptomic data, chronological age and phenotype data, in particular, data associated with indices of health.

Each subject can have an identifier, differentiating subjects from each other.

Protein data will include raw data or processed data. It can include any -omic data. However, transcriptomic data in particular will be included. This can include raw nucleic acid sequences. These can be converted into amino acid sequences. Aberrant proteins, in particular, are included. Raw data can include a measure, e.g., presence, absence or amount, of one or a plurality of aberrant proteins in each subject. Biological samples can be tested to provide a quantitative measure of one or a plurality of target proteins. In certain embodiments, the number of proteins in the training set can be at least any of 10, 50, 100, 200 or 500 different proteins.

The identity of the organ or tissue source can be included.

Phenotypic data from the subject also can be included, in particular for supervised learning. This typically will include the health status of the subject, e.g., with respect to the organ in question, but also overall health. In tests for senescence, the chronological age of the subject typically is included. Other phenotypic data derived from questionnaires is described herein.

Subjects typically will include a plurality of examples from different categories, e.g., health categories but also age categories. The age categories can span, for example, a single year, a five-year period, a decade or a just falling above or below a cutoff. So, for example, samples can be provided from individuals at each of a plurality of annual ages such as, any age from 0 to 120. Alternatively, samples can be grouped by decade such as single digits, 10s, 20s, 30s, 40s, 50s, 60s, 70s, 80s, 90s, or older. Alternatively, samples or subjects can be grouped as falling above or below a specified cutoff age. For example, the cutoff could be below 30 and 30 and above, or below 45 or 45 and above. Alternatively, a plurality of cutoffs can be used. So, for example, the samples can be grouped to include subjects below 30 and subjects about 50.

Alternatively, in the case of unsupervised learning, age labels may not be attached to each sample.

In certain embodiments, subject data can include data on a number of phenotypic characteristics of a subject. These can include indicators of health, disease and physiological functioning at the cell, tissue, organ, organ system or organismal level. Such information can function as tags or labels or categories that will be used to classify subjects. Other information can be transcriptomic data, e.g., blood transcriptomic data. Such data can include, at a raw level, quantitative measures of molecules in the transcriptome having particular nucleotide sequences. Such data can be processed to determine quantitative measures of aberrant forms, such as slice variants, sequence variants and expression levels.

Phenotypic data can include chronological age of a subject. In this way, individuals can be grouped into age cohorts, and differences in transcriptome features between cohorts can be identified. These differences function as biomarkers to classify an individual into a cohort, for example, determining “biological” age of a subject. This can be done with supervised or unsupervised learning methods. In turn, this method identifies particular aberrant proteins associated with senescence. These proteins can be the target of therapeutic intervention, as described herein.

Alternatively, subjects within a chronological age cohort can be compared with one another to determine transcriptome features associated with healthy and unhealthy states. This also can be done with supervised or unsupervised learning methods. For example, phenotypic labels can classify subjects based on liver health, heart health, brain health or immune function. A machine learning algorithm can identify features that can be used in a classification algorithm to distinguish individuals with healthy liver function versus unhealthy liver function.

2. Questionnaires

In certain embodiments, one or more questionnaires are used, where responses to the one or more questionnaires for the individual are used to partially or completely determine phenotype information for the individual, in particular, as related to biological conditions, for example biological conditions in an overall set of conditions. The questionnaire or questionnaires may include any suitable number of queries, for example, at least 5, 10, 15, 20, 25, 30, 35, 40, 45, 50, 55, 60, 65, 70, or over 70 questions. Responses to questions can be open-ended (e.g., the individual may provide a written response to a question without limit to content of the response, such as a written answer to a question such as “What are your health goals?”), questions with specific answers (e.g., “what medications do you take,” “what is your hip circumference in inches” and the like) or questions where the answer can be selected from a limited number of options, or a combination. Limited option questions include yes/no questions, true/false questions, questions that require selection of one or more response from a limited number of responses, which can be non-numerical responses (e.g., “what is your ethnicity,” with responses limited to “American Indian or Alaskan Native,” “Southeast Asian,” “South Asian,” “Asian,” “Black or African American,” “Native Hawaiian or other Pacific Islander,” “Caucasian/White,” “Hispanic or Latino,” or “Other”) or numerical responses (e.g., “How many cups of coffee do you drink each day,” with responses limited to 0, 1, 2, 3, 4, 5, 6, 7, 8, 9, or 10+; or “how often have you been bothered by a certain symptom (such as headache, or fatigue, or pain or aches in joints, etc.) in the past four weeks,” with the answers limited to “none,” “a little,” or “a lot,” etc.), or any other suitable question type that provides information useful in determining a biological condition.

Any suitable method of determining phenotype information from responses to the questionnaire(s), in particular, information regarding an individual set of biological conditions for an individual, may be used. For example, a first biological condition may be assessed by examining the responses to a first subset of questions in the questionnaire(s); the questions in a subset may be weighted so that answers to some questions count more than others. Specific responses to individual questions in the first subset may be assigned specific numerical values, which can be adjusted according to the weight of the question, then the numerical values for all responses in the first subset are totaled to give a phenotype score for the first biological condition. A similar procedure may be followed to assess a second, different biological condition in the individual, using a second subset of questions in the questionnaire(s) to provide a phenotype score for the second biological condition; the second subset of questions may be the same as or different from the first subset. The process may be repeated for any suitable number of biological conditions; when biological conditions for an individual are determined from an overall set of biological conditions, the upper limit will, of course, be the number of biological conditions in the overall set (or fewer, if some of the biological conditions in the overall set are mutually exclusive). Thus, the process can be repeated for at least 2, 3, 4, 5, 6, 7, 8, 9, 10, 12, 15, 17, 20, 22, 25, 30, or 35 conditions, to produce the same number of phenotype scores; each different biological condition is assessed with reference to responses to its own specific subset of questions, which may be the same as or different from subsets for other biological conditions. Questions may belong to more than one subset for more than one biological condition, or may belong to only one subset.

Phenotypic information can be obtained, for example, from subject responses to questionnaires, or from a chat bot that interacts with the subject through natural language conversations. Such questionnaires may gather information on traits such as age, sex, weight, blood type, headaches, faintness, dizziness, insomnia, watery or itchy eyes, swollen, red or sticky eyelids, bags or dark circles under eyes, blurred or tunnel vision (not including near or far-sightedness), itchy ears, earaches, ear infections, drainage from ear, ringing in ears, hearing loss, stuffy nose, sinus problems, hay fever, sneezing attacks, excessive mucus formation, chronic coughing, gagging, need to clear throat, sore throat, hoarseness, loss of voice, swollen or discolored tongue, gums or lips, canker sores, acne, hives, rashes, dry skin, hair loss, flushing, hot flashes, excessive sweating, irregular or skipped heartbeat, rapid or pounding heartbeat, chest pain, chest congestion, asthma, bronchitis, shortness of breath, difficulty breathing, bloated feeling, nausea, vomiting, diarrhea, constipation, belching, passing gas, heartburn, intestinal/stomach pain, pain or aches in joints, arthritis, stiffness or limitation of movement, pain or aches in muscles, feeling of weakness or tiredness, binge eating/drinking, craving certain foods, excessive weight, compulsive eating, water retention, underweight, fatigue, sluggishness, apathy, lethargy, hyperactivity, restlessness, poor memory, confusion, poor comprehension, poor concentration, poor physical coordination, difficulty in making decisions, stuttering or stammering, slurred speech, learning disabilities, poor physical coordination or clumsiness, numbness or tingling in hands or feet, mood swings, anxiety, fear or nervousness, anger, irritability or aggressiveness, sadness or depression, frequent illness such as colds, frequent or urgent urination, genital itch or discharge, decreased libido and PMS. Phenotypic information can be collected all in a single session, in several sessions involving a small number of questions at each session, and over weeks, months or years, creating a ‘longitudinal’ view of the subject's phenotype. Each of these can be a biological condition.

B. Model Building

Using these methods, models can be built to identify protein biomarkers for unhealthy states. In such methods, the training dataset will include data on proteins from healthy and unhealthy samples. The protein data can include any “-omic” data, e.g., any or all of genomic data, transcriptomic data or proteomic data. This can include both qualitative data, such as sequence data, as well as quantitative data, such as amounts of mRNA or proteins.

Machine learning can identify the features (proteins and/or amounts) associated with unhealthy states. These features can function as biomarkers for detection of unhealthy states. They also can function as targets for therapeutic interventions, as described below.

Methods for generating models to predict biological age can comprise the following operations. A dataset as described above is provided. The dataset includes, for each of a plurality of subjects, quantitative measures of aberrant proteins. The data set is used as a training dataset to train a machine learning algorithm to produce one or more models that predict biological age of a subject based on the aberrant protein.

1. Machine Learning Algorithms

Learning algorithms are trained on the training dataset to generate models that predict the biological age of an individual based on quantitative measures of aberrant proteins. Predicted biological age can be translated into recommendations to the subject about actions to take, such the methods described below to eliminate aberrant cells.

The machine learning algorithm can be any suitable supervised machine learning algorithm, parametric or non-parametric. Unsupervised machine learning methods also can be used. In supervised methods values for the categorical variable to be inferred are provided for each object in the dataset. In unsupervised methods, such values are not included in the dataset. Inference may be determined by, for example clustering data in inferring the cluster to which a subject belongs.

Machine learning algorithms include, without limitation, artificial neural networks (e.g., back propagation networks), decision trees (e.g., recursive partitioning processes, CART), random forests, discriminant analyses (e.g., Bayesian classifier or Fischer analysis), linear classifiers (e.g., multiple linear regression (MLR), partial least squares (PLS) regression, principal components regression (PCR)), mixed or random-effects models, non-parametric classifiers (e.g., k-nearest neighbors), support vector machines, and ensemble methods (e.g., bagging, boosting).

In artificial neural networks and interconnected group of nodes organized into a plurality of layers of modes. These may include an input layer one or more hidden layers and an output layer. Each node inputs may be summed e.g. based on their weights.

Support vector machines draw hyperplanes in multidimensional space to divide objects in the training dataset into categories.

C. Model Application

Using a classifier as described above, and operator can classify or infer a health state, including senescence in a subject based on aberrant protein data from the subject. The classifier can classify a condition according to any classification scheme useful to the operator. This can include, for example, a binary classification, such as present or absent or, according to a numeric scale, such as a 1-10 scale. Biological age can also be predicted based on a continuous (regression) model, which can be more accurate if the training data allow for that.

Classifications can be provided to a subject for example, in the form of recommendations. In one embodiment, the recommendations include a positive recommendation to undertake a therapeutic intervention, as described herein.

Classification involves providing a test dataset from a subject that includes as variables, those used by the classifier to make the inference. This can be the same data as used to generate the classifier, or a subject of that data. The classifier operates on that test dataset to classify the subject or subject sample according to whatever categorical variable is of interest. This could be state of health of a particular organ or organs, or age of a subject or degree of senescence of a subject of a certain age.

In certain embodiments the classifier will include a plurality of aberrant protein forms. The classifier may or may not be a linear model, e.g., of the form aX+bY+cZ=N, wherein a, b and c are measured amounts of aberrant proteins X, Y and Z. The classifier may require, for example, support vector machine analysis. For example, the inference model may perform a pattern recognition in which an aberrant protein profile lies on a scale between normal and abnormal, with various profiles tending more toward normal or toward abnormal. Thus, the classifier may indicate a confidence level that the profile is normal or abnormal.

The classifier or model may generate a single diagnostic number which functions as the model. Classifying senescence can involve determining whether the diagnostic number is above or below a threshold (“diagnostic level”). For example, the diagnostic number can be an absolute or relative amount of an aberrant protein. The threshold can be determined, for example, based on a certain deviation of the diagnostic number above normal individuals. A measure of central tendency, such as mean, median or mode, of diagnostic numbers can be determined in a statistically significant number of normal and abnormal individuals. A cutoff above normal amounts can be selected as a diagnostic level of senescence. That number can be, for example, a certain degree of deviation from the measure of central tendency, such as variance or standard deviation. In one embodiment the measure of deviation is a Z score or number of standard deviations from the normal average.

### VI. Systems

Also provided herein are systems comprising a computer. Such systems can be used for, among other things, executing learning algorithms, executing classification algorithms to predict biological age. Computer systems can include a central processing unit (also referred to as a CPU or a processor) memory (e.g., random-access memory, read-only memory, flash memory), communication interface for communicating with one or more other systems, and peripheral devices.

Such systems can be connected through a communications network to the Internet. The communications network can be any available network that connects to the Internet. The communication network can utilize, for example, a high-speed transmission network including, without limitation, Digital Subscriber Line (DSL), Cable Modem, Fiber, Wireless, Satellite and, Broadband over Powerlines (BPL).

Systems can include non-transitory computer readable medium that can contain machine-executable code that, upon execution by a computer processor, implements a method of the present disclosure.

VII. Methods of Eliminating Cells Expressing Aberrant Proteins from a Subject

Where a biopsy from a subject reveals the presence of cells expressing an aberrant protein, a therapeutic intervention can be administered to subjects to eliminate such cells. In certain embodiments, the cells expressing aberrant proteins that are targeted are nonmalignant cells. In certain embodiments a particular tissue or organ harboring the cells expressing the aberrant proteins are targeted.

Contemplated herein are several methods, or therapeutic interventions, of eliminating cells expressing aberrant proteins in a subject.

Cells present protein fragments on their surfaces through the MHC protein complex. Accordingly, methods of eliminating cells expressing aberrant proteins can target cells presenting fragments of aberrant proteins. Immunotherapy methods include, without limitation, biopharmaceuticals (such as small molecules, aptamers, antibodies, etc., that may or may not carry a cytotoxic payload), cell-based therapy (such as CAR T cell therapy and training of antigen presenting cells) and vaccination, including DNA vaccines. Cells that express aberrant proteins are eliminated by any one or more immunotherapy methods.

Such immunotherapeutic methods include, without limitation, the following. In certain embodiments an antibody drug conjugate can be delivered wherein the antibody drug conjugate comprises an antibody that specifically binds to a fragment of a protein comprising an aberration. In another embodiment, car T cells that comprise chimeric antigen receptors comprising a binding portion that recognizes protein fragments comprising an aberration can be administered to a subject. Training of APC cells with the aberrant proteins may also be used as an immunotherapeutic approach.

A. Eliciting an Immune Response

One method of eliminating cells expressing aberrant proteins involves eliciting an immune response in the subject to the aberrant protein. This can be done by administering to the subject a portion of the protein that contains the aberration. This portion will function as an antigen or vaccine, mobilizing the host immune response against the protein. The immune system will, in turn, attack and kill cells expressing such fragments on their surface.

B. Antibody Therapy/Antibody Drug Conjugates

As used herein, the term “antibody-drug conjugate” refers to a conjugate between an antibody and a cytotoxic agent.

As used herein, the term “antibody” refers to an immunoglobulin that recognizes and specifically binds to a one or more target antigen(s), such as a protein, polypeptide, peptide, carbohydrate, polynucleotide, lipid or combinations thereof. This binding occurs through at least one antigen recognition site within the variable region of the immunoglobulin at one or more epitopes on the antigen. The variable region is most critical in binding specificity and affinity. As used herein, the term “antibody” encompasses intact polyclonal antibodies (immunoglobulins), intact monoclonal antibodies, antibody fragments, single chain Fv (scFv) mutants, multispecific antibodies, chimeric antibodies, humanized antibodies, human antibodies, hybrid antibodies, fusion proteins and any other immunoglobulin molecule comprising an antigen recognition site so long as the antibody exhibit the desired biological activity. Antibodies can be of (i) any of the five major classes of immunoglobulins, based on the identity of their heavy-chain constant domains—alpha (IgA), delta (IgD), epsilon (IgE), gamma (IgG) and mu (IgM), or (ii) subclasses (isotypes) thereof (E.g., IgG1, IgG2, IgG3, IgG4, IgA1 and IgA2). The lights chains can be either lambda or kappa.

Antibodies directed against aberrant proteins can be made by any method known in the art. Such antibodies are chosen to distinguish between aberrant forms of the protein in normal forms of the protein. For example, a fragment of a protein containing an aberration can be used to immunize an animal to generate antibodies.

Antibodies can be naked or conjugated to other molecules such as toxins, drugs, radioisotopes, chemotherapeutic agents, etc.

Cytotoxic agents include, for example, maytansinoid, auristatin, dolastatin, tubulysin, cryptophycin, pyrrolobenzodiazepine (PBD) dimer, indolinobenzodiazepine dimer, alpha-amanitin, trichothene, SN-38, duocarmycin, CC1065, calicheamicin, an enediyne antibiotic, taxane, doxorubicin derivatives, anthracycline and stereoisomers, azanofide, isosteres, analogs or derivatives thereof.

Antibodies against aberrant proteins are administered to a subject. These antibodies bind to cells expressing the aberrant proteins on their surfaces, resulting in cell death.

The method of claim 23, wherein the immunotherapy comprises administering a DNA vaccine comprising DNA encoding the aberrant protein or an aberrant fragment thereof. Immunotherapeutic.

Besides antibody, other affinity reagents (for example aptamers) or specific small molecules can also be used to target specific aberrant proteins or cells that produce them.

C. CAR T Cell Therapy

In another embodiment, cells expressing aberrant protein can be destroyed using CAR T cell therapy.

Chimeric antigen receptors (“CARs”) can include the following elements: (1) a signal peptide, (2) a target binding domain, (3) a hinge region (optional); (4) a transmembrane region and (5) an intracellular domain comprising a signal transduction domain, e.g., a CD3 signal transduction domain. Optionally, the CAR can further include any of: an Fc receptor signal transduction domain, a co-stimulatory (signal transduction) domain. That is, these optional elements can be included in addition to or instead of, independently any one or more of the other optional elements. At least one of the domains is heterologous to at least one of the other domains. That is, at least two domains are not found together in this arrangement in nature. In some embodiments, the domains of the CAR are all heterologous to each other.

In the present case the target binding domain comprises a polypeptide that recognizes and binds to an aberrant protein. Such polypeptides can comprise single chain antibodies made from antibodies that recognize the target polypeptide.

T cells from a subject are genetically engineered to express chimeric antigen receptors that target cells presenting aberrant proteins.

D. Training Antigen Presenting Cells

Antigen-presenting cells are cells that display antigen complexed with major histocompatibility complexes (MHCs) on their surfaces. Antigen presenting cells stimulate T cells to attack other cells that present these antigens on the surface. Accordingly, antigen presenting cells of the subject can be genetically engineered to express fragments of aberrant proteins on their surfaces. These genetically engineered cells can be administered to a subject in order to stimulate a T cell mediated immune response against cells of the subject that express the aberrant protein.

E. DNA Vaccines

DNA vaccines typically involve expression vectors engineered to express a protein target. The vaccine can be a plasmid vaccine or viral vaccine. Cells which take up the DNA will express the protein.

Accordingly, a method of eliminating cells expressing aberrant proteins comprises administering to a subject a DNA vaccine encoding the aberrant protein or fragment of it having an aberrant sequence. Once expressed in a subject the aberrant protein will provoke an immune response.

RNA vaccines may also be used to train the immune system to clear the aberrant cells from a tissue, organ, or organism.

F. Other Embodiments

In certain embodiments, aberrant proteins targeted for detection or destruction, aberrant cells targeted for elimination may be limited to certain specific classes as described herein. For example, in certain embodiments, the aberrant protein may not be a transmembrane protein or an anchored protein, that is, proteins located on the surface of the cell membrane and covalently attached to lipids embedded within it. An aberrant cell targeted may not be a pathological cell or a malignant cell, e.g., may be limited to senescent cells or non-malignant pathological cells. In some embodiments, aberrant proteins are not proteins that contribute to malignancy, for example, mutant forms of cell cycle regulating proteins, genes involved in apoptosis, oncogenes or tumor suppressor genes. In certain embodiments, aberrant proteins do not include passenger mutations in such genes, for example, accumulated mutations in oncogenes or tumor suppressor genes that do not, in and of themselves, cause malignancy.

### EXAMPLES

### I. Example 1

Blood samples and phenotypic data are collected from 1000 subjects belonging to different biological categories including chronological age, physical and mental health states, sex, etc. RNA from the samples is purified, reverse transcribed and sequenced to produce transcriptomic data. Phenotypic data and transcriptomic data are collected into a training data set.

The training dataset is used to train a machine learning algorithm to develop a classifier that categories a subject based on chronological age or based on health status at a particular chronological age. Age cohorts used for the classification include ages 18-28 and 60-70. Other age cohorts used for comparison range by decade: 20-29, 30-39, 40-49, 50-59, 60-69, 70-79, 80-89, 90-99, and 100+.

### II. Example 2

Phenotypic questionnaires and blood samples are taken from more than 500 subjects. The subjects belong to age categories from every decade between the teens and the 90s. They also belong to several health categories for a variety of organ systems and different health statuses in each age cohort, e.g., “robust” or “feeble”.

Transcriptomic data is generated from the blood samples and the transcriptomic data and phenotype data are used to train a machine learning algorithm to generate models. The models classify a subject according to age, health at particular ages, and general health both overall and for organ health (function).

Classifiers are examined to identify aberrant proteins that are used by the classifiers to make the classification. Several protein aberrations highly associated with senescence and with unhealthy states are identified.

### III. Example 3

A tissue biopsy from the liver of a human subject is collected. The tissue is homogenized, cells are lysed, and mRNA is isolated. mRNA is reverse transcribed to DNA. The DNA molecules are sequenced using high-throughput sequencing to produce nucleotide sequences. Nucleotide sequences are examined. It is determined that an aberrant structural protein associated with diminished cell function is present among the sequences in the form of a splice mutant. The aberrant protein is determined to be present at an amount consistent with diminished organ function.

An antibody that is specific for the aberrant form of the protein is administered to the subject in an amount effective to elicit an immune response by the subject against cells expressing the aberrant protein. This results in destruction of such cells in the liver.

A subsequent test of the subject shows improved liver function, whether stem cells naturally migrate to fill the gaps left by the aberrant cells, or the stem cells are introduced with a treatment.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one or more” and “one or a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description is not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A method comprising:
a) providing a tissue sample comprising aberrant cells from a subject; and
b) detecting, in the sample, the expression of at least one aberrant protein.

2. The method of claim 1, wherein the aberrant protein is an aberrant structural protein.

3. The method of claim 1, wherein the aberrant protein is selected from: a splice variant, a genetic variant, a glycosylation variant and a lipidation variant.

4. The method of claim 1, wherein the aberrant protein is encoded by a genetic variant selected from a single nucleotide polymorphism (SNP), an insertion, a deletion, a gene fusion, a transversion, and a gene truncation.

5. The method of claim 1, wherein the aberrant protein is a housekeeping protein.

6. The method of claim 1, wherein the aberrant protein is not encoded by an oncogene or a tumor suppressor gene.

7. The method of claim 1, wherein the aberrant protein is not a transmembrane protein.

8. The method of claim 1, wherein the subject is a human or a non-human animal.

9. The method of claim 1, wherein the tissue sample comprises a preponderance of senescent cells.

10. The method of claim 1, wherein the tissue is derived from an organ system selected from muscular system, digestive system, respiratory system, urinary system, reproductive system, endocrine system, circulatory system, nervous system, and integumentary system.

11. The method of claim 1, wherein the sample is a solid tissue biopsy sample (e.g., a needle aspirate), a bone marrow biopsy sample or a liquid biopsy sample.

12. The method of claim 1, wherein detecting comprises detecting an aberrant polypeptide.

13. The method of claim 12, wherein the aberrant polypeptide is detected by mass spectrometry.

14. The method of claim 13, wherein mass spectrometry comprises multiple reaction monitoring (MRM) mass spectrometry.

15. The method of claim 1, wherein detecting comprises detecting mRNA encoding the aberrant protein.

16. The method of claim 15, wherein detecting mRNA comprises sequencing cDNA prepared from the mRNA.

17. The method of claim 1, wherein detecting comprises sequencing genomic DNA encoding the protein.

18. The method of claim 1, wherein detecting comprises detecting a plurality of aberrant forms of a protein.

19. The method of claim 1, wherein detecting comprises detecting an aberrant form or forms of each of a plurality of proteins.

20. The method of claim 1, wherein detecting comprises determining a measure of the aberrant protein in the sample.

21. The method of claim 20, wherein the measure is selected from presence or absence, an absolute amount, and a relative amount (e.g., an amount of the aberrant protein compared to an amount of the normal protein).

22. The method of claim 20, further comprising determining whether the measure is above a threshold measure.

23. The method of claim 22, wherein the threshold is a function, e.g., a positive function, of the subject's chronological age.

24. The method of claim 1, wherein detecting comprises determining a fraction of cells in the tissue sample expressing the aberrant protein.

25. The method of claim 1, wherein no more than 1% of cells in the tissue are malignant.

26. The method of claim 1, further comprising:
c) inferring, from the quantitative measure, a biological age of the tissue.

27. The method of claim 1, further comprising:
c) outputting the quantitative measure to an electronic device accessible by the subject.

28. A method of eliminating, in a subject, aberrant cells expressing at least one aberrant protein comprising:
a) selecting a subject having aberrant cells that express the aberrant protein; and
b) administering to the subject an immunotherapy to eliminate the aberrant cells.

29. The method of claim 28, wherein the subject has aberrant cells in an organ system selected from muscular system, digestive system, respiratory system, urinary system, reproductive system, endocrine system, circulatory system, nervous system, and integumentary system.

30. The method of claim 28, wherein the subject is identified by the method of claim 1.

31. The method of claim 28, wherein a biopsy sample from the subject detects the presence of aberrant cells expressing the aberrant protein.

32. The method of claim 28, wherein the immunotherapy comprises administering to the subject an antibody that binds to a fragment of a protein, wherein the fragment comprises an aberrant amino acid sequence presented by an MHC molecule on a surface of the cell.

33. The method of claim 32, wherein the antibody is an antibody-drug conjugate.

34. The method of claim 28, wherein the immunotherapy comprises training of APC cells with the aberrant proteins.

35. The method of claim 28, wherein the immunotherapy comprises eliciting an immune response by administering the aberrant protein form to the subject.

36. The method of claim 28, wherein the immunotherapy comprises administering a DNA vaccine comprising DNA encoding the aberrant protein or an aberrant fragment thereof.

37. The method of claim 28, wherein the immunotherapy comprises administering to the subject a CAR-T cell that recognizes a cell expressing the aberrant form of the protein.

38. The method of claim 28, wherein the aberrant cell is a senescent cell or a pathological cell.

39. The method of claim 28, wherein the aberrant cell is not a malignant cell.

40. The method of claim 28, wherein the aberrant protein is not a transmembrane protein, membrane-anchored protein or a protein encoded by an oncogene or a tumor suppressor gene.

41. An article comprising:
a) one or a plurality of solid supports, each solid support comprising a nucleic acid probe that binds to a nucleic acid having a nucleotide sequence of a senescence-associated polypeptide.

42. A composition comprising:
a) one or a plurality of pairs of oligonucleotide primers, each pair of primers adapted for amplifying a nucleic acid having a nucleotide sequence of a senescence-associated polypeptide.

43. A composition comprising:
a) for each of one or a plurality of senescence-associated polypeptides, a stable isotope standard polypeptide.

44. A method comprising:
a) providing a set of biological samples from each of plurality of subjects wherein the subjects comprise a plurality of subjects from each of a plurality of different functional states;
b) performing -omic analysis on each of the biological sample to produce an -omic data training dataset; and
c) training a machine learning algorithm on the -omic data training data set to generate a mapping function that infers an aberrant functional state from the -omic data.

45. The method of claim 44, wherein the functional state is a stage of senescence.

46. The method of claim 44, wherein the stages of senescence span a time period of at least one year, at least 3 years, at least 5 years, at least 10 years, at least 20 years, at least 30 years, at least 40 years, at least 50 years, at least 60 years, at least 70 years, at least 80 years, at least 90 years, or at least 100 years.

47. The method of claim 46, wherein, within the span of time, the plurality of ages are separated by no more than 6 months, one year, five years, ten years or 20 years.

48. The method of claim 46, wherein, at least 3 ages are separated by no more than 3 years, 6 years, 9 years or 15 years.

49. The method of claim 44, wherein each of the ages comprise at least any of 10, 25, 50, 100 or 200 subjects.

50. The method of claim 44, wherein the different functional states comprise different health states.

51. The method of claim 50, wherein the health states comprise healthy and unhealthy functioning of an organ or an organ system.

52. The method of claim 44, wherein the biological samples comprise samples from a tissue selected from epithelial, connective, nervous and muscle tissue.

53. The method of claim 44, wherein the -omic data is selected from genomic, epigenomic, transcriptomic, proteomic, metabolomic, lipidomic, glycomic, immunomic, phenomic and exposomic.

54. The method of claim 53, wherein the -omic data is blood transcriptomic data.

55. The method of claim 44, wherein performing -omic analysis comprises sequencing nucleic acids in the sample.

56. The method of claim 55, wherein the nucleic acids comprise genomic DNA and/or mitochondrial DNA.

57. The method of claim 55, wherein the nucleic acids comprise RNA.

58. The method of claim 57, wherein the RNA comprises mRNA.

59. The method of claim 44, wherein performing -omic analysis comprises bifulfite treatment of nucleic acids and sequencing of the bisulfite-treated nucleic acids.

60. The method of claim 44, wherein performing -omic analysis comprises separating and identifying proteins in the sample.

61. The method of claim 60, wherein separating and identifying is performed by mass spectrometry.

62. The method of claim 44, wherein performing -omic analysis comprises sequencing polysaccharides in the sample.

63. The method of claim 44, wherein performing -omic analysis comprises measuring metabolites in the sample.

64. The method of claim 44, wherein the mapping function is a classifier.

65. The method of claim 44, wherein the mapping function is a regressor.

66. The method of claim 44, wherein the mapping function uses measures of aberrant nucleic acid sequences.

67. The method of claim 44, wherein the mapping function uses measures of aberrant proteins.

68. The method of claim 44, wherein the mapping function uses measures of aberrant complex carbohydrates.

69. The method of claim 44, wherein the classifier uses gene expression information to infer functional state.

70. A method comprising:
a) providing a biological sample from a subject;
b) performing -omic analysis on the biological sample to produce an -omic data test dataset;
c) executing a mapping function on the -omic data to infer a chronological age based on the -omic data; and
d) outputting the inferred age to an electronic device accessible by the subject.

71. A method comprising:
a) providing a biological sample from a subject;
b) performing -omic analysis on the biological sample to produce an -omic data test dataset;
c) executing a mapping function on the -omic data to infer function or dysfunction of one or more organs in the subject based on the -omic data;
d) outputting the inferred age to an electronic device accessible by the subject.

72. A method comprising:
a) providing a chronological age and biological sample from a subject;
b) performing -omic analysis on the biological sample to produce an -omic data test dataset;
c) executing a mapping function on the -omic data to infer an age based on the -omic data;
d) determining that a difference between the chronological age and the inferred age is above a predetermined threshold; and
e) administering to the subject an (organ-specific) immunotherapy to destroy cells expressing the aberrant proteins.
